(19) United States
(12) Patent Application Publication
(10) Pub. No.: US 2017/0332966 A1
(43) Pub. Date: Nov. 23, 2017

(54) FIXATION METHOD FOR A NASAL SEPTUM SENSOR FOR MEASURING MEDICAL PARAMETERS

(71) Applicant: KONINKLIJKE PHILIPS NV., EINDHOVEN (NL)

(72) Inventors: Egbertus Reinier JACOBS, OVERLOON (NL); Wouter Herman PEETERS, WAALRE (NL); Johannes Wilhelmus WEEKAMP, BEEK EN DONK (NL); Johannes Henricus Maria VAN ROOSMALEN, SINT OEDENRODE (US); Rick BEZEMER, AMSTERDAM (NL); Igor Wilhelmus Franciscus PAULUSSEN, NUENEN (NL); Toeno VAN DER SAR, EINDHOVEN (NL)

(21) Appl. No.: 15/522,371

(22) PCT Filed: Oct. 16, 2015
(86) PCT No.: PCT/IB2015/057954

§ 371 Date: Apr. 27, 2017

Related U.S. Application Data
(60) Provisional application No. 62/069,937, filed on Oct. 29, 2014.

(51) Int. Cl. A61B 5/00; A61B 5/024; A61B 5/1455; A61M 16/06

(52) U.S. Cl. CPC A61B 5/6819; A61B 5/14552; A61B 5/02416; A61M 16/06

(57) ABSTRACT

When measuring SpO2 in a patient, a nasal oximeter is inserted into the patient's nose and comprises a source pad (14) and a detector pad (18) that are positioned at a predetermined location on either side of the nasal septum. The source pad and detector pad are biased toward each other to provide a clamping force on the nasal septum sufficient to permit SpO2 measurement without causing tissue necrosis. Clamping force is supplied by means of a spring type device, an expandable material, or the like.

The present invention finds application in pulse oximetry measurement systems and methods. However, it will be appreciated that the described techniques may also find application in other vital sign measurement systems, other sensor placement techniques, and the like.

In the intensive care unit (ICU) or the operating room (OR), there is a need to evaluate certain parameters of the human body. As two of them are of particular interest, being photoplethysmography (PPG) and pulse oximetry (SpO2), they need to be measured in a reproducible way. Normally, these sensors are very susceptible to movement and the signal responds accordingly, making them very unreliable.

In addition to the motion issue, there is also the issue that conventional PPG/SpO2 sensors do not perform well in patients with low perfusion conditions, especially when the sensor is positioned on a fingertip. Low-perfusion conditions typically occur in the first 24 hours after ICU admission, and are often associated with low cardiac output, hypothermia, hypovolemia, hypotension, shock, and influence by vasopressor therapy. One conventional approach to addressing this issue involves a pulse oximeter that applied on the forehead. However, this pulse oximeter placement is not ideal.

An alternative site for the pulse oximeter is the nasal septum, which has various advantages including that the nasal septum remains perfused in very low-perfusion conditions since it is perfused by the ethmoidal arteries, which branch from the internal carotid artery. Furthermore, the nasal cavity is optically shielded from the environment, which dramatically reduces optical noise coming from, e.g., hospital lighting systems.

One disadvantage of the nasal septum as a measurement site is that the site is not easily accessible as it is hidden rather deeply inside the nasal cavity. Conventional approaches are deficient in affixing the pulse oximeter on the nasal septum in a stable manner.

One approach to mounting a nasal septum pulse oximeter relates to mounting a pulse oximeter on a nasal cannula (U.S. Pat. No. 7,024,235). However, this approach does not provide a stable measurement, as the sensor is positioned low in the nasal cavity and applies no pressure on the septum. The nasal cannula itself is not a stable mounting position. Another disadvantage of this approach is that the sensor cannot be mounted without a nasal cannula. Therefore, patients must be wearing a nasal cannula in this approach.

The present application provides new and improved systems and methods that provide a pulse oximeter that is stably mountable to the nasal septum and that is exerting a limited and well-controlled pressure onto the nasal septum, thereby overcoming the above-referenced problems and others.

In accordance with one aspect, a pulse oximeter device for oximetry sensing across a nasal septum comprises a flat, flexible adhesive portion coupled to a first flexible member and a second flexible member, a source pad that emits light and is coupled to the first flexible member, and a detector pad that detects the light transmitted through the nasal septum by the source pad and is coupled to the second flexible member. When affixing pulse oximeter to a given patient, the adhesive portion is folded to have a curvature approximating the curvature of an outer surface of a given patient's nose. The adhesive portion comprises an adhesive on a patient-facing surface thereof that adheres the adhesive portion to the patient's nose when the source pad and the detector pad are aligned at a predetermined position.

According to another aspect, an under-the-nose pulse oximeter device for oximetry sensing across a nasal septum comprises a spring portion, a source pad that emits light and is coupled to the spring portion, and a detector pad that detects the light transmitted through the nasal septum by the source pad and is coupled to the spring portion. The spring portion, when manipulated, causes the source pad and the detector pad to be biased away from each other during insertion into a nose. The spring portion, when released after positioning the source pad and the detector pad at a predetermined location along the nasal septum, causes the source pad and the detector pad to be biased toward each other thereby providing a clamping force on the nasal septum, where the clamping force is needed to push away venous blood from the vascular bed thereby improving the accuracy of the pulse oximetry measurement.

According to another aspect, an internalized pulse oximeter device for oximetry sensing across a nasal septum comprises a source pad that emits light and is coupled to a first securing component, and a detector pad that detects the light transmitted through the nasal septum by the source pad and is coupled to a second securing component. The first and second securing components are compressible during insertion of the source pad and the detector pad into the nose, and expandable when positioned at a predetermined position along the nasal septum thereby providing a clamping force that biases the source pad and the detector pad toward each other and against the nasal septum at a predetermined location.

According to another aspect, a pulse oximeter device for oximetry sensing on a nasal septum comprises a flat, flexible adhesive portion coupled to a first member and a second member, a source that emits light, and a detector. When affixing pulse oximeter to a given patient, the adhesive portion is folded to have a curvature approximating the curvature of an outer surface of a given patient's nose. The adhesive portion comprises an adhesive on a patient-facing surface thereof that adheres the adhesive portion to the patient's nose when the source pad and the detector are aligned at a predetermined position.

One advantage is that the source pad and detector pad are clamped to the nasal septum with sufficient force to permit SpO2 measurement.

Another advantage is that the clamping force is sufficiently low to prevent decubitus or tissue necrosis.

Another advantage is that the pulse oximeter is mounted stably on the nose to prevent signal artifacts caused by movement of the patient.

Still further advantages of the subject innovation will be appreciated by those of ordinary skill in the art upon reading and understand the following detailed description.

The drawings are only for purposes of illustrating various aspects and are not to be construed as limiting.

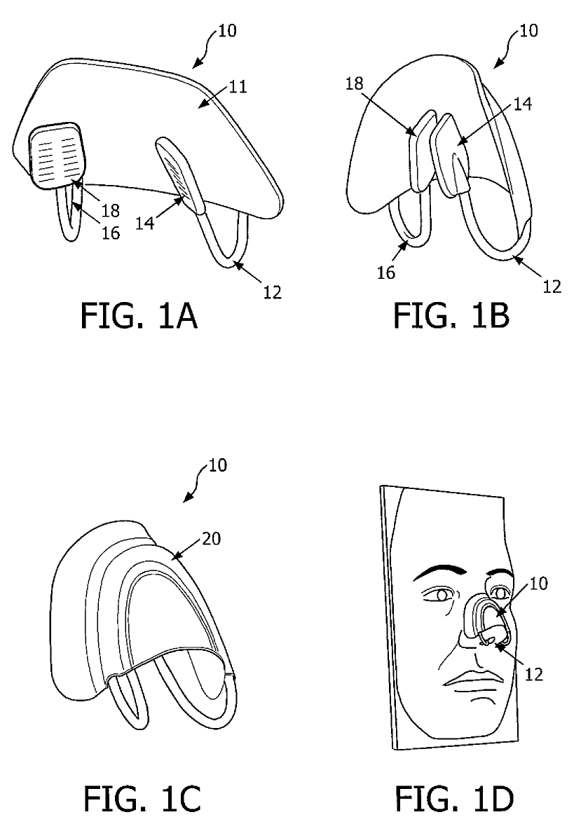

FIG. 1A shows a nasal septum pulse oximeter device, which can be affixed to a patient's nasal septum in a stable manner, in an unfixed or unattached state, in accordance with one or more aspects described herein.

FIG. 1B shows the oximeter assembly in a folded state such as occurs when the assembly is affixed to a curved surface, such as a nose.

FIG. 1C shows the outward surface of the oximeter assembly, which comprises a channel through which a flexible member runs.

FIG. 1D illustrates the oximeter assembly coupled to a patient's nose.

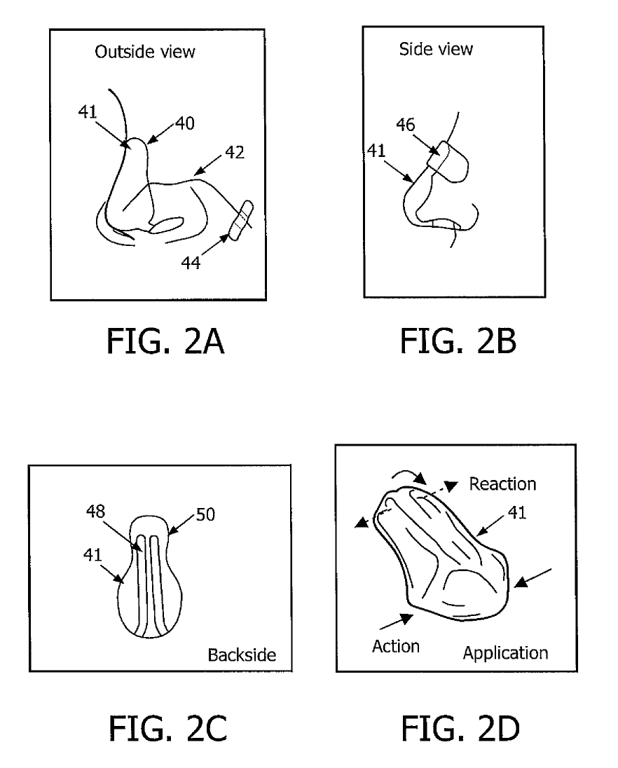

FIG. 2A illustrates an outer view of the pulse oximeter device, comprising a support portion, wire leads extending from the patient's nostrils, and a fixation dressing that secures the wires to the patient's skin so that the wires do not pull on the oximeter device.

FIG. 2B shows a side view of the pulse oximeter device in which fixation plaster has been placed over the support portion at the bridge of the nose to stabilize the oximeter device.

FIG. 2C shows a rear view of the oximeter device, in which a LED or source portion and a detector or sensor portion of the device can be seen.

FIG. 2D shows how the oximeter device is applied.

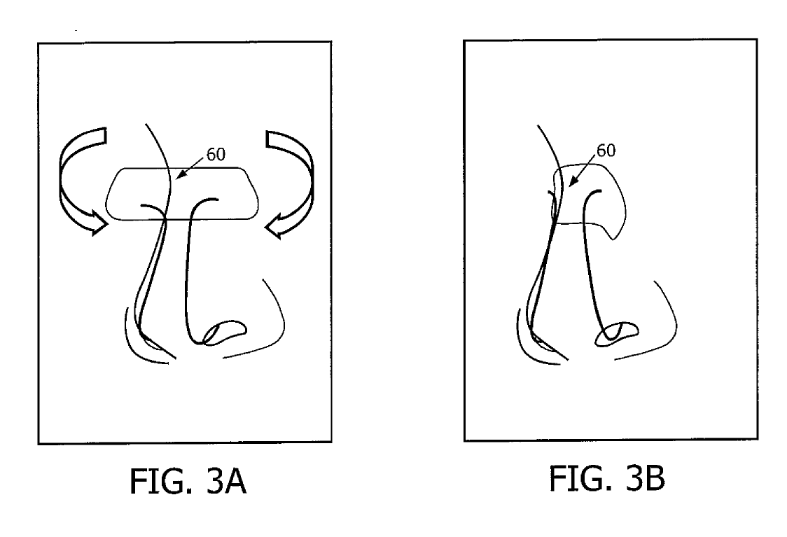

FIG. 3A illustrates a plaster bending approach to affixing the support portion of the oximeter device, in which the fixation plaster material is shown in an unbent state, with arrows indicating the direction of bending to be applied.

FIG. 3B illustrates the fixation plaster in the bent or applied state.

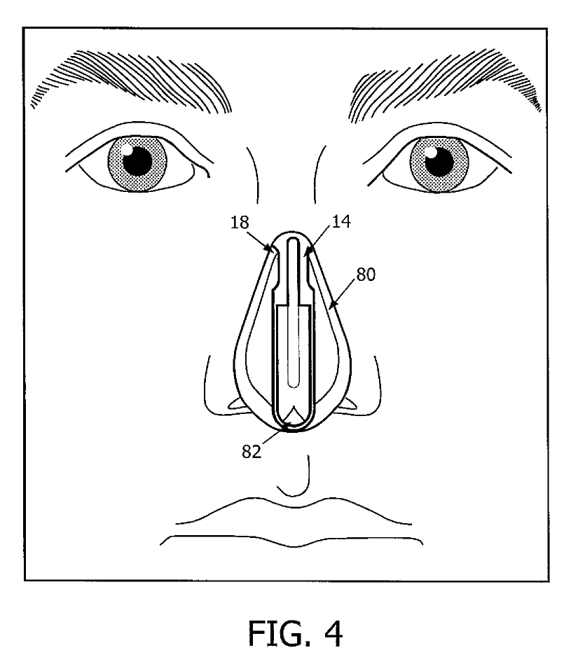

FIG. 4 illustrates a leaf spring clip oximeter device, in accordance with one or more aspects described herein.

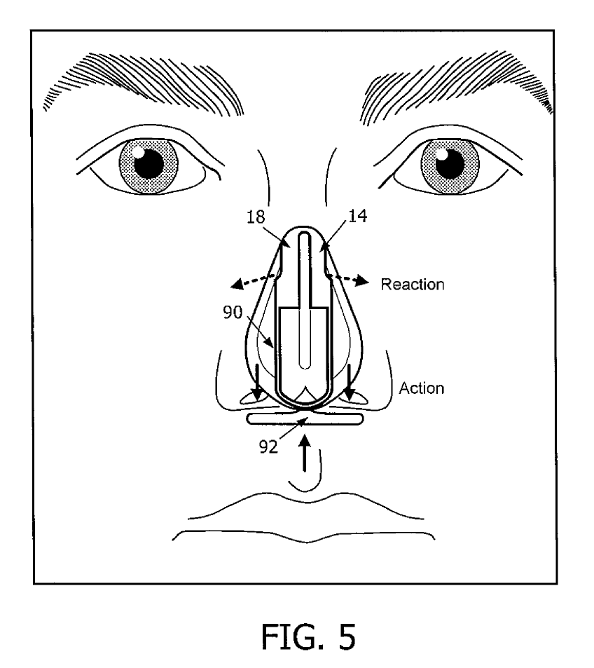

FIG. 5 illustrates a crossing spring oximeter device, in accordance with one or more aspects described herein.

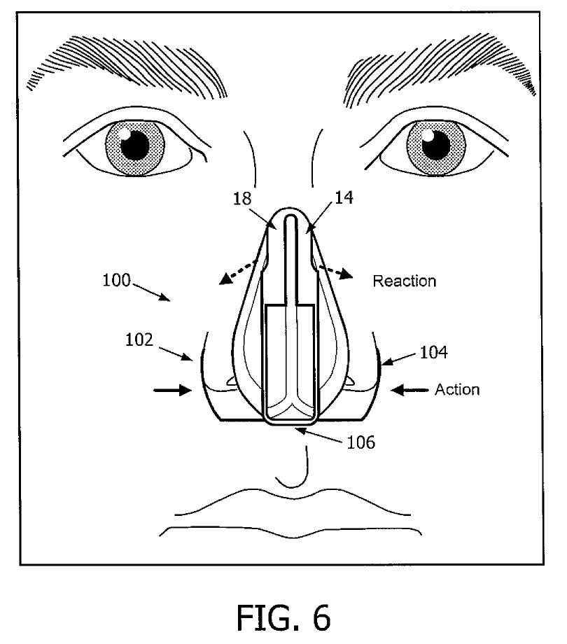

FIG. 6 illustrates a hinged clip oximeter device, in accordance with one or more aspects described herein.

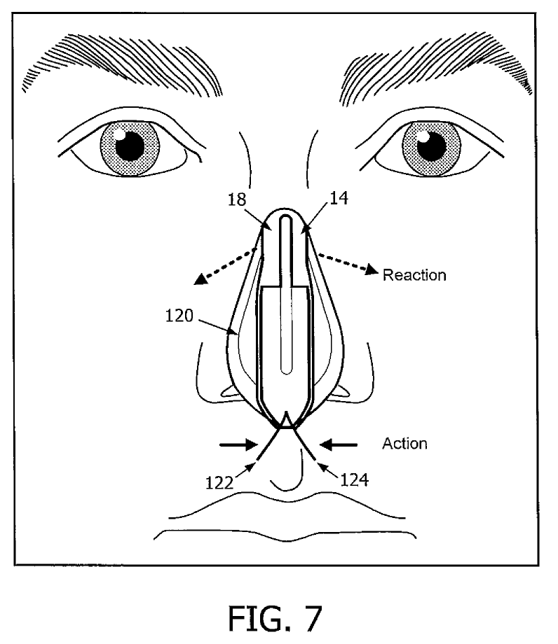

FIG. 7 illustrates a clothes pin type oximeter device, in accordance with one or more aspects described herein.

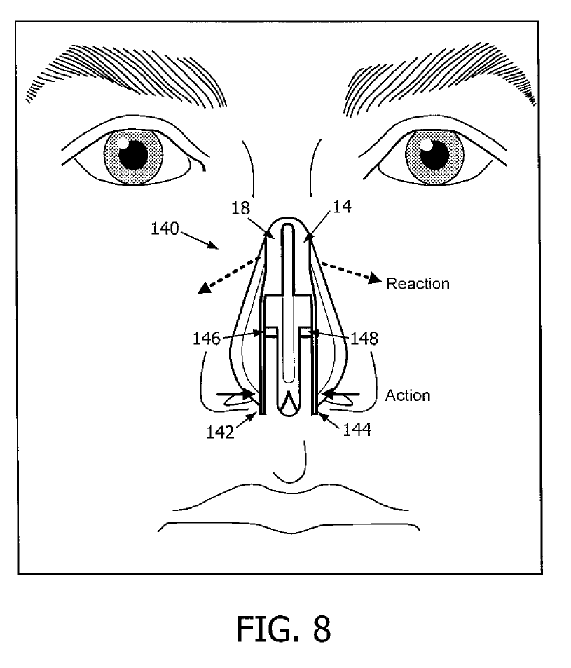

FIG. 8 illustrates an internal hinge oximeter device, in accordance with one or more aspects described herein.

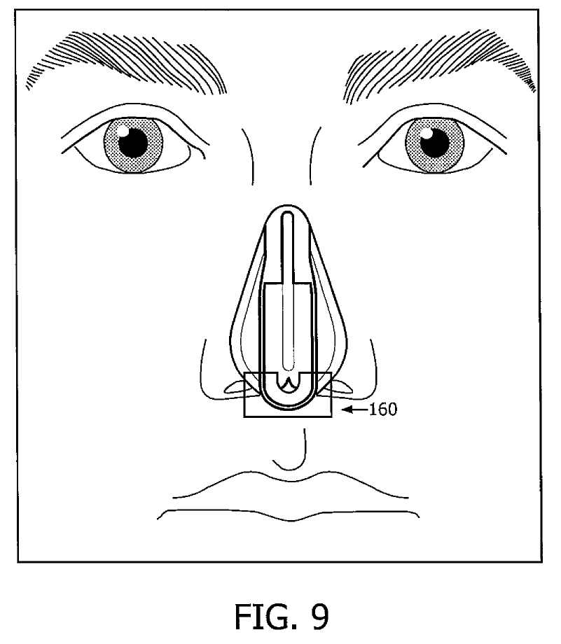

FIG. 9 illustrates an insertion tool that facilitates opening the herein-described spring-loaded oximeter devices.

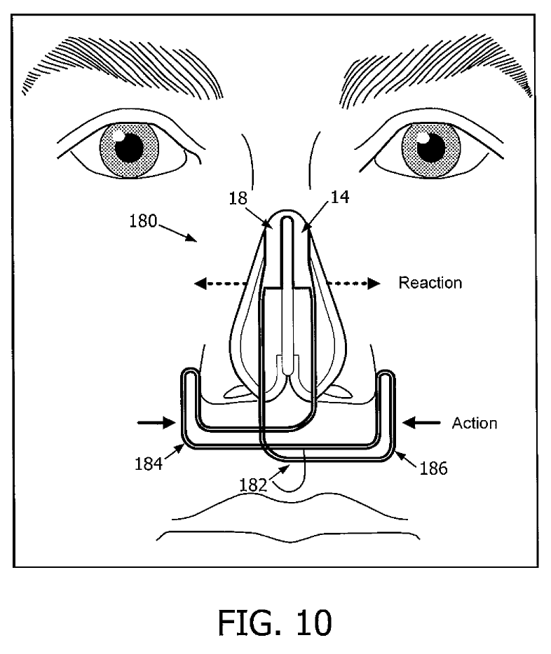

FIG. 10 illustrates a long leaf spring oximeter device, in accordance with one or more aspects described herein.

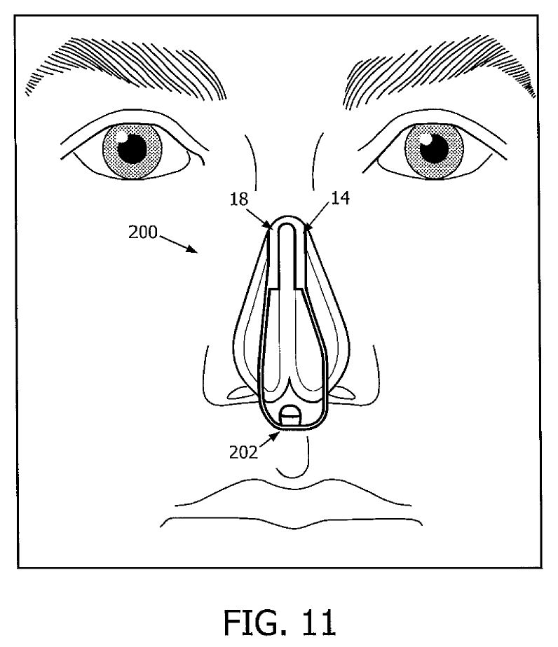

FIG. 11 illustrates a wired clip oximeter that provides two separate tunable forces for clamping to the nasal septum, in accordance with various aspects described herein.

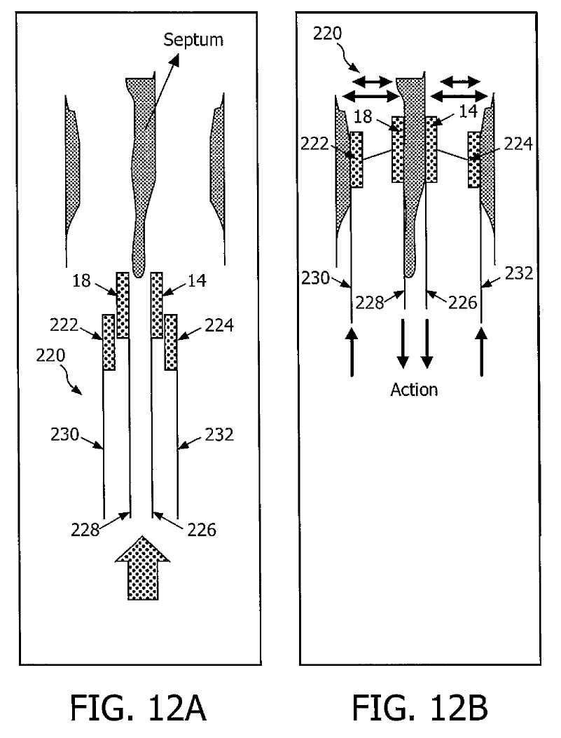

FIG. 12A illustrates a double hinge oximeter device for insertion into a nose, in accordance with various aspects described herein.

FIG. 12B shows the double hinge oximeter device in an inserted position.

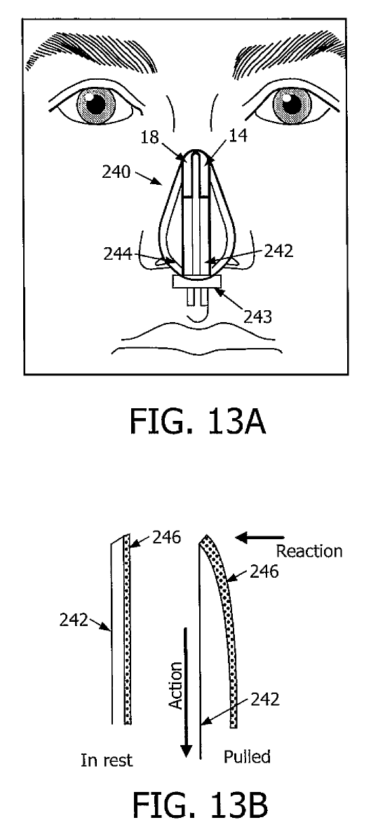

FIG. 13A illustrates a catheter type oximeter device, in accordance with one or more aspects described herein.

FIG. 13B shows the flexible outer post at rest and after tension has been applied to the wire.

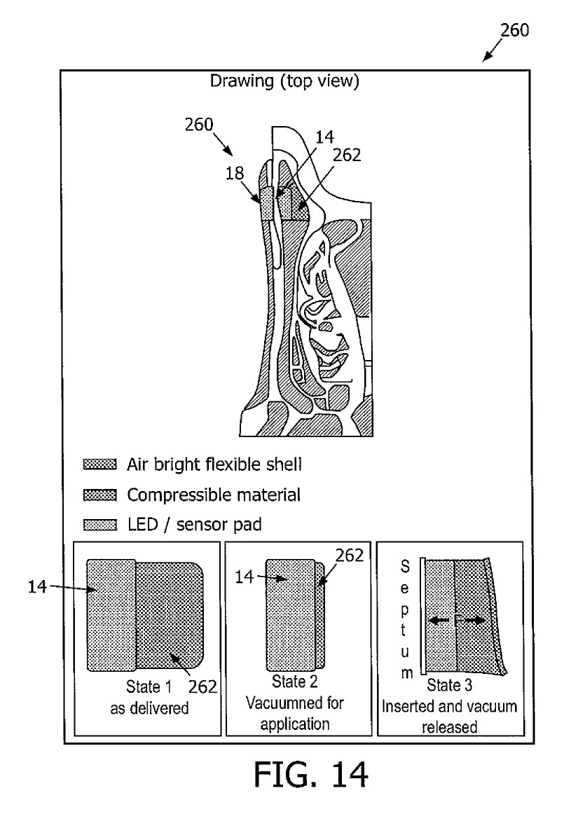

FIG. 14 illustrates a vacuum type oximeter device, in accordance with one or more aspects described herein.

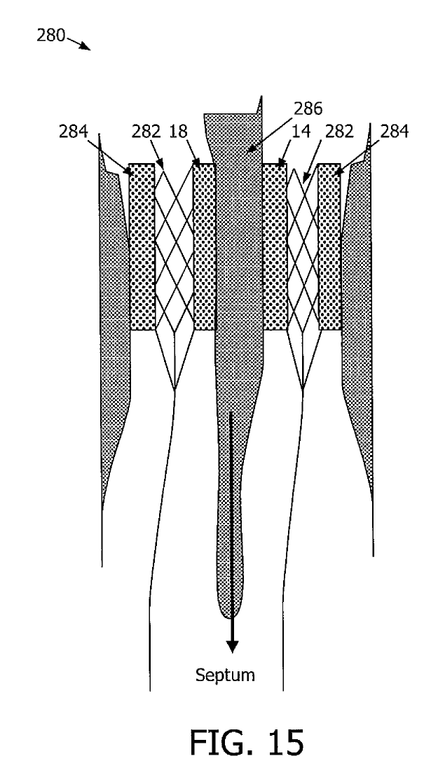

FIG. 15 shows a stent-like clamping oximeter device, in accordance with one or more aspects described herein.

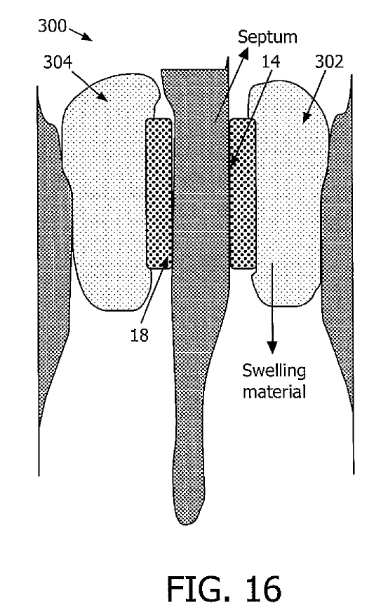

FIG. 16 shows a swelling-type oximeter device, in accordance with one or more aspects described herein.

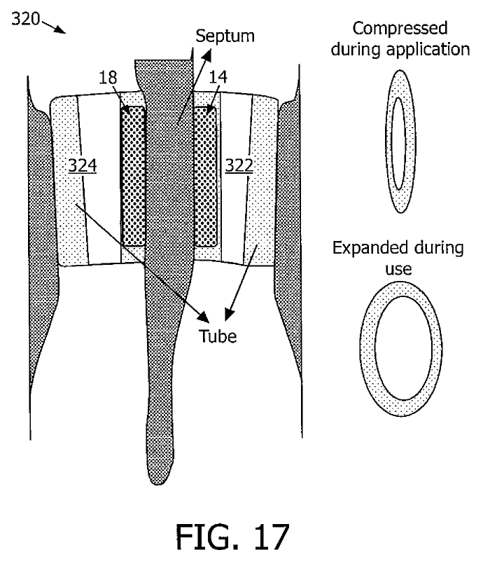

FIG. 17 illustrates a tube clamping type oximeter device, in accordance with one or more aspects described herein.

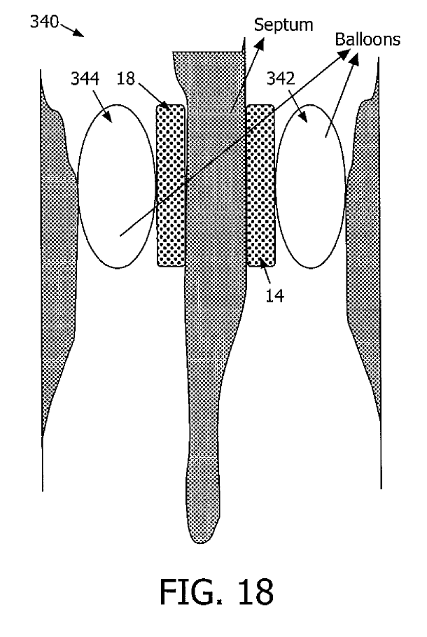

FIG. 18 illustrates a balloon clamping oximeter device, in accordance with one or more aspects described herein.

The described systems and methods overcome the above-mentioned problems by providing systems and methods for affixing devices tightly to the nasal septum without applying excessive pressure on the tissue. The nasal septum has several benefits for measuring SpO2 and PPG signals, as it is a very thin and well perfused part of the body and can be probed in transmission geometry, contrasting the reflection geometry that is utilized by forehead probes. The nasal septum as a measurement site also has the benefit of being one of the last well-perfused sites when a patient enters a state of shock. Accordingly, several systems and methods of stably fixating an oximetry sensor to the nasal septum are described herein.

FIGS. 1A-1D illustrate various views of a nasal septum pulse oximeter 10 that can be affixed to a patient's nasal septum in a stable manner, in accordance with one or more aspects described herein. FIG. 1A shows the oximeter 10 in an unfixed or unattached state. The oximeter comprises an adhesive portion 11 coupled to a first flexible member 12 to which a source pad 14 is coupled, and a second flexible member 16 to which a sensor or detector pad 18 is coupled. The adhesive portion in the example of FIG. 1 is an adhesive patch or the like, although other means of fixation (e.g., a clip) to a surface (e.g., a human nose or the like) are contemplated. The first flexible member 12 guides the electric wires that conduct the current through the one or two or more LEDs that are part of the source pad 14. The second flexible member 16 guides the electric wires that conduct the photodetection current that is generated in the detector pad 18.

FIG. 1B shows the oximeter assembly 10 in a folded state such as occurs when the assembly is affixed to a curved surface, such as a nose. As can be seen, the source pad 14 and the detector pad 18 are facing each other in a configuration that facilitates sensing SpO2.

FIG. 1C shows the outward surface of the oximeter assembly, which comprises a channel 20 through which a flexible member runs. In the illustrated embodiment, first and second flexible members 12, 16 comprise a continuous flexible structure that traverses the channel 20 and terminates in the flexible member portions 12, 16 that are respectively coupled to the source pad 14 and the detector pad 18.

FIG. 1D illustrates the oximeter assembly 10 coupled to a patient's nose. Only the adhesive patch 11 is visible, as the flexible members and respective source and detector pads are inserted into the patient's nose and positioned against the nasal septum.

With continued reference to FIGS. 1A-1D, the source pad 14 comprises light source of a predetermined surface area (e.g., 1 cm² or the like). In one embodiment, the source pad comprises a pair LEDs (not shown) or some other predetermined multiple of two LEDs, wherein one LED is red and the other (in each pair) is infrared. The detector pad 18 comprises a thin photodiode or a thin flexible photodiode (not shown) for measuring the transmitted light of the LEDs through the nasal septum. The adhesive patch 11 (or other fixation means, such as a clip) holds the two pads at a desired location, maintains the alignment, applies a desired amount of pressure or force, and fixates the oximeter assembly 10 onto/into the nose in a way that mitigates susceptibility to facial muscle movement.

The adhesive portion 11 comprises an adhesive layer (e.g., a plaster or glue or the like) and can be constructed in a flat orientation. Application of the patch is performed as follows. The patch is removed from a backing (e.g., a waxed paper or the like) and the pads slide into the nostrils by an upwards motion. When at the desired position, a protective foil (not shown) is removed from the adhesive layer on the patient-facing side of the adhesive portion. In one embodiment, plaster is folded around the nasal bridge and held in place by the deformable part of the wire inside the plaster. This action also provides the force on the nasal septum. The adhesive layer on the inner surface of the adhesive portion 11 sticks onto the nose, securing the location. Once the oximeter assembly is affixed, the measurement can commence.

The force on the septum is controlled by the stiffness of the flexible members on which the detector pad and detector pad are mounted. The clamping force on the septum can be sufficiently low such that no necrosis or mucosal damage occurs, because the fixation of the sensor is secured by means of the adhesive plaster, which allows to reduce the clamping force, thereby reducing the clamping force to a level that does not cause decubitus or necrosis. The flexibility of the source and detector pads ensures that the delicate mucosal tissue is not damaged.

In one embodiment, the folding of the plaster does not affect the separation of the flexible members 12 and 16, and a separation between the source pad 14 and the detector pad 18 is secured by a removable solid portion between the two flexible members. The separation is sufficiently large to allow insertion of the source pad 14 and the detector pad 18 into the two nostrils. After insertion, the removable portion can be removed from the device such that the permanent spring force of the flexible members is released and transferred via the source pad and the detector pad onto the nasal septum. After this force is released, the measurement can commence.

The length of the flexible members 12, 16 can be selected to be long enough to reach the upper part of the Kiesselbach's plexus where perfusion is resistant to severe cases of low perfusion. The thinness of the source and detector pads ensures that the pads fit in the small space that is available high up in the nasal cavity (e.g., less than 2 mm).

The length of the flexible members 12, 16 can be selected to be rather short, e.g., about 2 mm to 2 cm, such that insertion is minimally inconvenient for the patient, and the caregiver can visually verify whether the sensor pad are positioned correctly.

Stability of the oximeter assembly 10 is ensured because the adhesive plaster is attached to the nasal bridge, which is a very rigid part of the body. There are no muscles under the skin that influence the positioning. The bended flexible members 12, 16 transfer the stable mounting provided by the adhesive plaster on the outside of the nose, towards the nasal septum, which is in contrast to a known method based on fixation on a nasal cannula.

In one embodiment, the flexible members are padded with a deformable material in order to prevent uncomfortable pressure points on the rims of the nostrils. The padded material softens the contact force between the flexible members and the rims of the nostrils. The deformable material can have a slow response to any applied force, for example in the order of 10 seconds to 10 minutes. At the moment the sensor is applied, the deformable material is not yet deformed and may exert an unwanted force on the nose; after the relaxation time of the material, e.g., 10 seconds to 10 minutes, the deformable padding will deform until it follows the natural shape of the nose such that any unwanted force on the nose is fully disappeared. The deformable material can be any flexible material, like for example silicone. The deformable material can be partly cured silicone in a bag-type cushion circumvention. The deformable material can also be a highly viscous gel that is enclosed in a bag-type of cushion circumvention. The deformable material can also be a material where the fluidity or viscosity is temperature dependent.

Because the adhesive portion 11 is foldable, the oximeter assembly can fit on any nose. The orientation of the tip of the nose varies between patients, as well as the width of the nostrils. The bended flexible members 12, 16 are arranged a geometry that circumvents this variability, since the flexible members are hindered by neither the shape of the tip of the nose nor the width of the nostrils.

The area underneath the nostrils is also kept clear such that nasal tubing can still be applied when the oximeter assembly is in place. The flexible members are narrow enough that they do not occlude the nostrils, and the length of the flexible members positions the detector pads between at a predetermined distance along the nasal septum (e.g., between 0.2 cm and 4.5 cm, or the like). Since the lower 1 cm of the septum is unimpeded by the oximeter assembly 10, there is ample of room to introduce nasal tubing for feeding or ventilation of the patient. The foldable adhesive portion is also very thin so that a ventilation mask can be placed over the patient's nose and mouth without air leakage.

As the pulse oximetry measurement is very susceptible to movement, the described oximeter device embodiments ensure that the oximeter device is fixated to the body in a very stable manner. Stable mounting represents a challenge for a sensor that needs to reach the nasal septum as a target site, as there are various challenges that need to be overcome. For instance, the pressure on the septum needs to be just right as the venous blood system of the nasal cavity needs to be gently “squeezed” to obtain solely arterial pulsation in between the source and detector pads. Arterial pulsation results in a difference in blood volume in between the source and detector pad, and from this a PPG signal can be derived. Excessive force can result in decubitus or tissue necrosis (when pressed over a longer period of time), and insufficient pressure might give and unreliable signal.

Additionally, the location of the source and detector pad in the nose can also influence the measurement. The position considered to give the best results is the Kiesselbach's plexus, as this tissue is mainly supplied by the anterior ethmoidal artery originating from the brain. It is therefore desirable that the source and detector pad be positioned high up in the nasal cavity (e.g., up to 4.5 cm from the bottom of the bridge 22 in between the nostrils). This location provides a usable vascular bed for determining PPG/SpO2 under low perfusion conditions (e.g., large amount of blood loss, shock or vasorelaxation during surgery).

Moreover, the measurement is very susceptible to movement and for this reason placement on the facial muscular system may be undesirable when affixing an oximeter. Any movement of the lips or cheek can cause large motion artifacts if the sensor is fixated on skin covering these muscles. The described embodiment is not affected by this source of motion artefacts, as the pulse oximeter device is fixated by an adhesive patch covering the nasal bridge. Another consideration is that there is high variability in the shape of human noses, making a “one size fits all” challenging. Compatibility with other interventional systems:

During surgery and/or in an ICU, several other systems are used for treating or monitoring patients. Accordingly the described fixation methods and systems are configured in such a way as not to interfere with these systems. In order not to interfere with ventilation masks, for instance, the fixation means can be made flat on the outside of the nose to prevent air leakage underneath the edges of the ventilation mask. Ideally, the fixation method must be on the outside of the nose, flat or close to the nasal tip Also, to allow for the use oxygen tubes and feeding tubes, the entrance of the nostrils should be left unblocked by the fixation method. This can be achieved by making the source pad and the detector pad sufficiently thin, or the source pad and detector pad should be place sufficiently high along the nasal septum in order not to block the lower part.

In another embodiment, the source and detector are disposed on the same pad, and a reflector pad is disposed on the second member to reflect light from the source back across the nasal septum to the first pad where the light is detected by the detector.

In yet another embodiment, the first and second members are rigid and mutually connected by a spring (not shown) that biases the first and second members toward each other.

FIGS. 2A-2D illustrate a nasal oximeter device 40 for stably affixing the oximeter to the patient's nose, in accordance with one or more aspects described herein.

FIG. 2A illustrates an outer view of the oximeter device 40, comprising a support portion 41, wire leads 42 extending from the patient's nostrils, and a fixation dressing 44 that secures the wires to the patient's skin so that the wires do not pull on the oximeter device.

FIG. 2B shows a side view of the oximeter device 40 in which fixation plaster 46 (or other suitable fixation means) has been placed over the support portion 41 at the bridge of the nose to stabilize the oximeter device.

FIG. 2C shows a rear view of the oximeter device 40, in which an LED or source portion 48 and a detector or sensor portion 50 of the device can be seen.

FIG. 2D shows how the oximeter device is applied. By pinching the bottom of the device, the flexible support portion biases the source portion 48 and the sensor portion 50 away from each other for insertion into the nose. Once at the desired position, the bottom of the support portion is released and the source portion and sensor portion grip the nasal septum. The clamping force and the force on the septum can be decoupled, as the clamping is done towards the nasal bridge. Although these forces are decoupled, the material stiffness and design determine how much force is applied to the septum during the measurement. If the force is too great, the force on the septum can be decreased by using support pads, after which the pressure can be more easily regulated by material and design.

FIGS. 3A and 3B illustrate a plaster bending approach to affixing the support portion (see, e.g., FIGS. 2A-2D) of the oximeter device. In FIG. 3A, the fixation plaster material 60 is shown in an unbent state, with arrows indicating the direction of bending to be applied. In FIG. 3B, the fixation plaster 60 is shown in the bent state. In one embodiment, the plaster is combined with a pre-shaped body (not shown) which converts the bending action (when the plaster is bent over the nasal bridge) into a sensor pressure onto the septum. The force for holding the sensor is decoupled from the septum force. The septum force can be predetermined. Additionally, no applicator is needed and, as part of the oximeter device is outside the nose, it can easily be removed.

The features described with regard to FIGS. 1-3B above relate to exterior-mounted embodiments for positioning oximeter devices on the nasal septum. FIGS. 4-11 describe aspects related to “under-the-nose” embodiments for positioning oximeter devices on the nasal septum.

FIG. 4 illustrates a leaf spring clip oximeter device 80, in accordance with one or more aspects described herein. The leaf spring clip oximeter device comprises a leaf spring portion 82 that is coupled to each of a source pad 14 and a detector pad 18, such as are described with regard to various embodiments herein. The leaf spring portion can be coated in, e.g., silicone or some other material for safety and comfort, and can be tuned to obtain predetermined spring characteristics. Additionally, the oximeter device 80 can be adjusted using separate pads (not shown) lower in the nose than the source and detector pads, to reduce force on the septum.

FIG. 5 illustrates a crossing spring oximeter device 90, in accordance with one or more aspects described herein. The oximeter device has a large spring leaf 92 for operation of the device below the nostrils. The outer edges of the spring leaf 92 are squeezed downward while the center is pushed upward as indicated by the solid arrows to bias the source pad 14 and detector pad 18 away from each other as indicated by the dashed arrows during insertion of the device, and then released to grip the nasal septum at the desired location. Force on the spring can be tuned according to the characteristics of the leaf spring and/or material. This embodiment also allows for single handed insertion of the device. This embodiment has a longer spring length relative to other embodiments for better control of the force. Additionally, the shape enables one hand actuation by pressing in the manner indicated by the solid arrows areas in the drawing.

FIG. 6 illustrates a hinged clip oximeter device 100, in accordance with one or more aspects described herein. The hinged clip device comprises a pair of wings 102, 104 that, when squeezed together or biased toward each other about a hinge point 106 cause the source pad 14 and the detector pad 18 to be biased away from each other to permit insertion of the oximeter device into the nose. Releasing the wings causes source pad and detector pad to travel toward each other to apply pressure against the septum and hold the device in place. This embodiment facilitates manipulation of the device for insertion and placement, and provides tunable pressure on the septum. In this manner, the device 100 utilizes a hinge to ease the handling of the device. Manipulation is then moved to the side of the nose. The bulky handles are however very susceptible for external disturbances, like accidentally bumping against the handles. Also for this concept there is no decoupling between the septum force and the clamping force for keeping the system on the nose.

FIG. 7 illustrates a clothes pin type oximeter device 120, in accordance with one or more aspects described herein. The clothes pin oximeter device 120 comprises a pair of wings 122, 124, which are biased toward each other about a hinge or spring 126 to cause the source pad 14 and the detector pad 18 to be biased away from each other to permit insertion of the oximeter device into the nose. Releasing the wings causes source pad and detector pad to travel toward each other to apply pressure against the septum and hold the device in place. This embodiment facilitates manipulation of the device for insertion and placement, and provides tunable pressure on the septum.

FIG. 8 illustrates an internal hinge oximeter device 140, in accordance with one or more aspects described herein. The internal hinge oximeter device 140 comprises a pair of wings 142, 144, which are biased toward each other about a respective hinge or spring 146, 148 to cause the source pad 14 and the detector pad 18 to be biased away from each other to permit insertion of the oximeter device into the nose. Releasing the wings causes source pad and detector pad to travel toward each other to apply pressure against the septum and hold the device in place. This embodiment facilitates manipulation of the device for insertion and placement, and provides tunable pressure on the septum. The internal hinge oximeter device facilitates providing shorter handles outside the nostrils and therefore less movement of the sensor by accidentally bumping into it. In one embodiment, the internal hinge oximeter device includes support pads on the hinge holder and so that only the hinge force is used to provide pressure on the septum.

FIG. 9 illustrates an insertion tool 160 that facilitates opening the herein-described spring-loaded oximeter devices. The tool 160 is provided with a given oximeter device and is employed to insert an position the device, at which point the tool is removed in order to release the spring and provide clamping force on the septum.

FIG. 10 illustrates a long leaf spring oximeter device 180, in accordance with one or more aspects described herein. The long leaf spring clip oximeter device comprises a long leaf spring portion 182 that is coupled to each of a source pad 14 and a detector pad 18, such as are described with regard to various embodiments herein. The long leaf spring oximeter device 180 comprises a pair of wings 184, 186, which are biased toward each other to cause the source pad 14 and the detector pad 18 to be biased away from each other to permit insertion of the oximeter device into the nose. Releasing the wings causes source pad and detector pad to travel toward each other to apply pressure against the septum and hold the device in place. This embodiment facilitates manipulation of the device for insertion and placement, and provides tunable pressure on the septum. The long leaf spring oximeter device can be coated in, e.g., silicone or some other material for safety and comfort, and can be tuned to obtain predetermined spring characteristics.

FIG. 11 illustrates a wired clip oximeter 200 that provides two separate tunable forces for clamping to the nasal septum, in accordance with various aspects described herein. Force on the septum can be reduced while clamping at the nose bridge can be maintained. The wired clip oximeter comprises a spring 202 that clamps the source pad 14 and the detector pad 18 towards the nasal bridge and a decoupled septum force for applying the needed pressure for the measurement. As those two forces are completely decoupled, a higher clamping force can be applied in order to fixate the sensor.

FIGS. 12A-18 relate to internalized oximeter device arrangements, such that the oximeter device is wholly within the nose with only wire leads protruding therefrom, if present. While FIGS. 4-11 relate to spring-type embodiments for supplying clamping force to the nasal septum, FIGS. 12A-18 relate to expansion-type embodiments wherein an expandable or compressible securing component or material is coupled to each of the source pad and the detector pad, and inserted into the nose in a compressed state and then permitted to expand to secure the pads to the nasal septum.

FIG. 12A illustrates a double hinge oximeter device 220 for insertion into a nose, in accordance with various aspects described herein. A lever function is provided that clamps the pads to the septum, permitting the clamping force to be adjusted and readjusted externally. The double hinge device comprises a source pad 14 and a detector pad 18 hingeably coupled to respective outer pads 222 and 224. Each of the source pad 14, the detector pad 18, and the outer pads 222 and 224 is coupled to a respective wire or wand 226, 228, 230, 232, which are manipulated to position the oximeter device in the nose and adjust the force applied to the nasal septum.

FIG. 12B shows the double hinge oximeter device 220 in an inserted position. The double hinge (or double wedge) can be used internally and the adjustment of the force can be regulated from the outside.

FIG. 13A illustrates a catheter type oximeter device 240, in accordance with one or more aspects described herein. Catheters are operated by pull wires to steer the tip in the right direction. The force is converted from a pulling force at the bottom of the system to a lateral force only at the tip. The catheter type oximeter device 240 comprises a pair of inner flexible wires 242, 244 respectively coupled to flexible outer posts 246 (FIG. 13B) coupled to the source pad 14 and the detector pad 18 to cause the source pad 14 and the detector pad 18 to be biased toward the nasal septum once inserted into the nose when tension is applied to the wires via a tensioning device 245.

FIG. 13B shows the flexible outer post 246 at rest and after tension has been applied to the wire 242. A similar flexible post is coupled to the detector pad 18 and wire 244 (FIG. 13A).

FIG. 14 illustrates a vacuum type oximeter device 260, in accordance with one or more aspects described herein. The vacuum oximeter device comprises a source pad 14 and a detector pad 18, each having a compressible material portion 262 adapted to be compressed by vacuum for insertion into the nasal passages. Once inserted (e.g., using an insertion tool or the like) and positioned, the vacuum is terminated and the compressible material portion expands to hold the source pad and detector pads in place against the nasal septum. In one embodiment, the oximeter device also comprises sensors for detecting blood pressure. According to another embodiment, an applicator is used to ensure the correct positioning of the pads relative to one another. The applicator remains, while the pads are supplied with a vacuum applied to the compressible material. Then the pads are inserted by means of the applicator to the correct position in the nasal cavity and the vacuum is released. The pads then expand and fixate into the nostril and the applicator is then removed. The extraction is done in a reverse manner, by re-applying the vacuum and pulling the wire/tube to extract the pads from each nostril.

FIG. 15 shows a stent-like clamping oximeter device 280, in accordance with one or more aspects described herein. A stent 282 is positioned between a source pad 14 and a brace pad 284 so that upon expansion of the stent the source pad is urged against the nasal septum 286. A similar arrangement is shown for the detector pad 18. Stent clamping is used to keep a passage open by employing a self-expanding wired stent. The stent is inserted in a compressed state into a tube structure and then released by pushing the stent out of the tube. Stents are commonly used in hospitals for opening clogged arteries. The clamping force of the stent oximeter device can be regulated by the wire thickness. If an applicator is used, the alignment can be ensured as well. Another benefit is that the system is completely in the nose and only the wires protrude out of the nostrils. Removing the stent from the nostril can be performed by putting a tube over the wire and slide this into the nostril to compress the stent again.

FIG. 16 shows a swelling-type oximeter device 300, in accordance with one or more aspects described herein. In a manner similar to the vacuum type device of FIG. 14, the source pad 14 and detector pad 18 each comprise or are coupled to respective expandable material portions 302, 304. The expandable material is in a compressed state during insertion and placement, and thereafter expands (e.g., in response to moisture in the nasal passages, moisture introduced manually or artificially thereto, etc.). The swelling type oximeter device 300 is disposed completely in the nostrils. Due to uptake of artificially introduced or native moisture, the swelling material expands and the device clamps into the nostril, and the force distribution is advantageously homogeneous.

FIG. 17 illustrates a tube clamping type oximeter device 320, in accordance with one or more aspects described herein. The tube clamping type oximeter device 320 comprises a pair of compressed tubes 322, 324 (e.g., silicone or some other material) that respectively encase the source pad 14 and the detector pad 18, which are released after placement in the nostrils. After release, the tube returns to its original shape, supplying pressure to the pads to bias the pads against the nasal septum. In one embodiment, an applicator is used to keep the tube in a flat state when inserted into the nostril. The tube is then released and returns to its original round tubular shape, providing the clamping force needed to keep the sensors in place. After removal of the applicator, only the wires protrude out of the nostrils.

FIG. 18 illustrates a balloon clamping oximeter device 340, in accordance with one or more aspects described herein. In a manner similar to the swelling type device of FIG. 16, the source pad 14 and detector pad 18 each comprise or are coupled to respective balloon portions 342, 344. The balloon portions are in a deflated state during insertion and placement, and thereafter inflated to bias the source and detector pads against the nasal septum. The device is extracted from the nostrils by deflating the balloons. An applicator can be employed to ensure proper alignment when inserting and positioning the device.

According to another embodiment, a moist-release clamping type oximeter device 360, in accordance with one or more aspects described herein. In this embodiment, a compressible material is applied to each of the source and detector pads. The compressible material is compressed and glued in place with a water-soluble epoxy or adhesive. Once in position in the nose, the moisture therein (or artificially introduced) dissolves the epoxy or adhesive and the compressible material expands to urge the source and detector pads against the nasal septum. An applicator can be employed for initial positioning of the source and detector pads.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. A pulse oximeter device for oximetry sensing across a nasal septum, comprising:
a flat, flexible adhesive portion coupled to a first flexible member and a second flexible member;
a source pad that emits light and is coupled to the first flexible member;
a detector pad that detects the light transmitted through the nasal septum by the source pad and is coupled to the second flexible member;
wherein, when affixing pulse oximeter to a given patient, the adhesive portion is folded to have a curvature approximating the curvature of an outer surface of a given patient's nose;
wherein the adhesive portion comprises an adhesive on a patient-facing surface thereof that adheres the adhesive portion to the patient's nose when the source pad and the detector pad are aligned at a predetermined position.

2. The pulse oximeter according to claim 1, wherein the first and second flexible members are opposite ends of a continuous flexible structure that traverses a channel on an outward-facing surface of the adhesive portion.

3. The pulse oximeter according to claim 1, wherein, the source pad comprises a thin, flexible, homogeneous light source of a predetermined surface area and further including at least one pair of LEDs, wherein in each pair of LEDs one LED is red and the other is infrared, and wherein the detector pad comprises a thin, photodiode for adapted to measure the transmitted light of the LEDs through the nasal septum.

4. The pulse oximeter according to claim 1, further comprising a plaster material that is affixed over the outer surface of the adhesive portion across the bridge of the nose and thereby further stabilizes the pulse oximeter.

5. The pulse oximeter according to claim 1, wherein the first and second flexible members have a length that extends from an entrance to the nose up to the Kiesselbach's plexus in the nose in order to position the source pad and detector pad there at.

6. The pulse oximeter according to claim 1, wherein the length of the flexible members positions the detector pads between at a distance of between 0.2 cm and 4.5 cm from the entrance of the nose along the nasal septum.

7. The pulse oximeter according to claim 1, wherein when in a folded state, the first and second members are biased toward each other such that the source pad and the detector pad apply a clamping force to both sides of the nasal septum.

8. The pulse oximeter according to claim 1, wherein the first and second members each further comprise a deformable pad portion positioned to contact, respectively, a rim of each nostril of the patient.

9-23. (canceled)

24. A pulse oximeter device for oximetry sensing on a nasal septum, comprising:
a flat, flexible adhesive portion coupled to a first member and a second member;
a source that emits light;
a detector;
wherein, when affixing pulse oximeter to a given patient, the adhesive portion is folded to have a curvature approximating the curvature of an outer surface of a given patient's nose;
wherein the adhesive portion comprises an adhesive on a patient-facing surface thereof that adheres the adhesive portion to the patient's nose when the source pad and the detector are aligned at a predetermined position.

25. The pulse oximeter device according to claim 24, further comprising a first pad coupled to the first member and on which the source and detector are disposed, and a second pad coupled to the second member and which reflects light from the source back across the nasal septum to the first pad where the light is detected by the detector.

26. The pulse oximeter device according to claim 24, wherein the first and second members are rigid and mutually connected by a spring that biases the first and second members toward each other.

27. The pulse oximeter according to claim 24, wherein the first and second flexible members are opposite ends of a continuous flexible structure that traverses a channel on an outward-facing surface of the adhesive portion.

28. The pulse oximeter according to claim 24, wherein the source pad comprises a thing, flexible, homogeneous light source of a predetermined surface area; and further including at least one pair of LEDs, wherein each pair of LEDs one LED is red and the other is infrared, and wherein the detector pad comprises a thin, photodiode for adapted to measure the transmitted light of the LEDs through the nasal septum.

29. The pulse oximeter according to claim 24, wherein when in a folded state, the first and second members are biased toward each other such that the source pad and the detector pad apply a clamping force to both sides of the nasal septum.

30. The pulse oximeter according to claim 24, wherein the first and second members each further comprise a deformable pad portion positioned to contact, respectively, a rim of each nostril of the patient.
